(19) United States
(12) Patent Application Publication
(10) Pub. No.: US 2008/0183223 A1
(43) Pub. Date: Jul. 31, 2008

(54) Hybrid jointed bone screw system

(76) Inventors: Dong M. Jeon, Draper, UT (US); Patrick D. Moore, West Jordan, UT (US)

Correspondence Address: MORRISS OBRYANT COMPAGNI, P.C., 734 EAST 200 SOUTH, SALT LAKE CITY, UT, 84102, US

(21) Appl. No.: 12/079,280

(22) Filed: Mar. 26, 2008

Related U.S. Application Data
(63) Continuation-in-part of application No. PCT/US05/39284, filed on Oct. 31, 2005.
(60) Provisional application No. 60/720,287, filed on Sep. 26, 2005.

(51) Int. Cl. A61B 17/56

(52) U.S. Cl. 606/305; 606/60; 606/329

(57) ABSTRACT

Assemblies, systems and components for a hybrid jointed bone screw system. A receiver member having an upper opening and a lower opening also includes an internal mating bone anchor interface channel at the bottom. A bone anchor is loaded into the lower internal mating bone anchor interface opening of the receiver member, and an anchor pin is “pushed” through the receiver member and through the bone anchor head to retain the bone anchor member. The bone anchor is capable of single-axial positioning throughout a range of motion of about 180 degrees. An elongated member may be placed in the upper channel of the receiver member and a compression retaining member applied via the upper opening. The compression retaining member presses down on the elongated member, which presses down on a floor of the upper channel, locking the elongated member between the retaining member and the receiver member floor.

### CROSS-REFERENCE TO RELATED APPLICATIONS

This application is a continuation-in-part of International Application PCT/US2005/039284 filed Oct. 31, 2005, which claims the benefit of U.S. Provisional Application No. 60/720,287, filed Sep. 26, 2005. The disclosures of each of these related applications are incorporated herein by reference in their entirety.

### TECHNICAL FIELD

The present invention relates to devices and implants used in osteosynthesis and other orthopedic surgical procedures such as devices for use in spinal surgery, and, in particular, to a posterior pedicle screw, connector/rod assembly which is implantable within a patient for stabilization of the spine. Specifically, the present invention contemplates a top loading bone anchor assembly capable of achieving multiple angular, as well as multiple spherical axial orientations with respect to an elongated member extending along bone tissue.

### BACKGROUND

Several techniques and systems have been developed for correcting and stabilizing damage or malformation of bones, especially the long bones and the spine. In one type of system, an elongated member such as a bendable rod is disposed longitudinally along a length of the bone(s). In spinal applications, the rod is preferably bent to correspond to the normal curvature of the spine in the particular region being instrumented. For example, the rod can be bent to form a normal kyphotic curvature for the thoracic region of the spine, or a lordotic curvature for the lumbar region. In accordance with such a system, the rod is engaged to various vertebrae along a length of the spinal column by way of a number of fixation elements. A variety of fixation elements can be provided which are configured to engage specific portions of the vertebra and other bones. For instance, one such fixation element is a hook that is configured to engage the laminae of the vertebra. Another very prevalent fixation element is a screw that can be threaded into various parts of the vertebrae or other bones.

In one typical spinal procedure utilizing a bendable rod, the rod is situated on opposite sides of the spine or spinous processes. A plurality of bone screws are threaded into a portion of several vertebral bodies, very frequently into the pedicles of these vertebrae. The rods are affixed to this plurality of bone screws to apply corrective and stabilizing forces to the spine.

One example of a rod-type spinal fixation system includes elongated rods and a variety of hooks, screws and bolts all configured to create a segmental construct throughout the spine. In one aspect of the system, the spinal rod is connected to the various vertebral fixation elements by way of an eyebolt. In this configuration, the fixation elements are engaged to the spinal rod laterally adjacent to the rod. In another aspect of the system, a variable angle screw is engaged to the spinal rod by way of an eyebolt. The variable angle screw allows pivoting of the bone screw in a single plane parallel to the plane of the spinal rod. Details of this variable angle screw can be found in U.S. Pat. No. 5,261,909 to Sutterlin et al. One goal achieved by the system is that the surgeon can apply vertebral fixation elements, such as a spinal hook or a bone screw, to the spine in appropriate anatomic positions. The system also allows the surgeon to easily engage a bent spinal rod to each of the fixation elements for final tightening.

Another rod-type fixation system provides a variety of fixation elements for engagement between an elongated rod and the spine. In one aspect of the system, the fixation elements themselves include a body that defines a slot within which the spinal rod is received. The slot includes a threaded bore into which a threaded plug is engaged to clamp the rod within the body of the fixation element. The system includes hooks and bone screws with this “open-back” configuration. Details of this technology can be found in U.S. Pat. No. 5,005,562.

On the other hand, these fixation elements of the system are capable only of pivoting about the spinal rod to achieve variable angular positions relative to the rod. While this limited range of relative angular positioning is acceptable for many spinal pathologies, many other cases require more creative orientation of a bone screw, for instance, relative to a spinal rod. Certain aspects of this problem are addressed by the variable angle screw of the system, as discussed in the '909 Patent. However, there is a need for a bone screw that is capable of angular orientation in multiple planes relative to the spinal rod as well as multiple spherical head orientations. Preferably, the bone screw axis is capable of various three dimensional orientations with respect to the spinal rod as well as three dimensional spherical axis orientation to the receiving (head) element of the devices' axial orientation of the bone engaging screw member. Screws of this type of angular orientation in multiple planes relative to the spinal rod have been referred to as poly-axial or multi-axial bone screws. One should note, as of yet, no screw systems have employed both angular orientation in multiple planes relative to the spinal rod and three dimensional spherical axis orientation to the receiving (head) element of the devices axial orientation of the bone engaging screw member. The use of both angular orientation in multiple planes relative to the spinal rod and three dimensional spherical axis orientation to the receiving (head) element of the device's axial orientation of the bone engaging screw member technology allows for virtually unlimited axial angulations of the bone engaging screw member as well as an ultra-low profile of the said device utilizing a minimum of components without sacrificing the security of the interfaces of the invention components.

Others have approached the solution to this problem with various poly-axial screw designs. For example, in U.S. Pat. No. 5,466,237 to Byrd et al., a bone screw is described which includes a spherical projection on the top of the bone screw. An externally threaded receiver member supports the bone screw and a spinal rod on top of the spherical projection. An outer nut is tightened onto the receiver member to press the spinal rod against the spherical projection to accommodate various angular orientations of the bone screw relative to the rod. While this particular approach utilizes a minimum of components, the security of the fixation of the bone screw to the rod is lacking. In other words, the engagement or fixation between the small spherical projection on the bone screw and the spinal rod is readily disrupted when the instrumentation is subjected to the high loads of the spine, particularly in the lumbar region.

In another approach shown in U.S. Pat. No. 4,946,458 to Harms et al., a spherical headed bone screw is supported within separate halves of a receiver member. The bottom of the halves are held together by a retaining ring. The top of the receiver halves are compressed about the bone screw by nuts threaded onto a threaded spinal rod. In another approach taken by Harms et al., in U.S. Pat. No. 5,207,678, a receiver member is flexibly connected about a partially spherical head of a bone screw. Conical nuts on opposite sides of the receiver member are threaded onto a threaded rod passing through the receiver. As the conical nuts are threaded toward each other, the receiver member flexibly compresses around the head of the bone screw to clamp the bone screw in its variable angular position. One detriment of the systems in the two Harms et al. patents is that the spinal rod must be threaded in order to accept the compression nuts. It is known that threading rods can tend to weaken the rods in the face of severe spinal loads. Moreover, the design of the bone screws in the '458 and '678 Patents require a multiplicity of parts and are fairly complicated to achieve complete fixation of the bone screw.

A further approach illustrated in U.S. Pat. No. 5,797,911 to Sherman et al. is to provide a U-shaped holder through the top of which a bone fastener topped with a crown member is loaded. The holder accommodates a rod in a channel above the crown member and a compression member above the rod. The compression member presses on the rod and crown member to lock the fastener against the holder in any of a number of angles in three dimensions with respect to the rod. This approach has proven to be quite effective in addressing the above-identified problems. However, it does not permit bottom-loading of the fastener. Additionally, the holder is somewhat bulky in order to accommodate the other structural components.

Yet a further approach is shown in U.S. Pat. No. 5,733,285 to Errico et al. in which a holder is provided with a tapered and colletted portion at the bottom into which a bone fastener head is inserted. A sleeve is provided that slides down around the colletted portion to crush-lock the colletted portion around the head of the bone fastener. This apparatus is believed to be relatively bulky and difficult to manipulate given the external sliding locking mechanism. It is further dependent on the fit of the external sleeve and the relative strength of the collet and its bending and crushing portions for secure locking of the bone fastener head.

There is therefore a need remaining in the industry for an ultra-low profile, hybrid jointed bone anchor that can be readily and securely engaged to an elongated member of any configuration—i.e., smooth, roughened, knurled or even threaded—which achieves greatly improved angulations of the bone anchor, improved strength, and reduced size, including profile and bulk, of the components used to engage the bone anchor to the elongated member in any of a variety of angular orientations.

### SUMMARY

In one illustrative embodiment of a hybrid jointed bone screw system in accordance with the present invention, a receiver member has an upper opening and a lower opening, which may not form a continuous opening. An internal mating bone anchor interface channel is disposed at the bottom of the receiver member. The head of a bone anchor may be loaded into the lower internal mating bone anchor interface opening of the receiver member, and an anchor pin pushed through the receiver member and through the bone anchor head to retain the bone anchor member.

The bone anchor is capable of single-axial positioning throughout a range of motion of about 180 degrees. Generally, the hybrid bone screw system is designed to include two different axial relationship positions (0 degrees and 90 degrees apart) of the bone anchor member with respect to the receiver member, and specifically, this axial relationship to the axial placement of an elongated rod member.

An elongated member may be placed in an upper channel of the receiver member through the upper opening to contact a channel “floor.” A compression retaining member may be applied via the upper opening to press down on the elongated rod member, which presses down on the channel floor, thus locking the elongated rod member in the receiver member.

Since such a single elongated member/receiver member construct, in itself does not provide the necessary axial “locking” required for design parameters, the necessary “locking” parameter may be accomplished by alternatively placing one each of an elongated member/receiver member construct on either side of the spine, at each vertebral body to be fused. This unique apparatus placement locks the total apparatus construct into one solid unit.

Additional embodiments, examples, advantages, and objects of the present invention will be apparent to those of ordinary skill in the art from the following specification.

### DESCRIPTION OF THE DRAWINGS

It will be appreciated by those of ordinary skill in the art that the elements depicted in the various drawings are not to scale, but are for illustrative purposes only. The nature of the present invention, as well as other embodiments of the present invention may be more clearly understood by reference to the following detailed description of the invention, to the appended claims, and to the several drawings attached hereto.

FIGS. 1A and 1B are side and front views of one embodiment of a hybrid joint bone anchor assembly in accordance with the principles of the present invention.

FIGS. 2A and 2B are exploded front and side views of the components of the embodiment depicted in FIGS. 1A and 1B.

FIGS. 3A and 3B are front and side views of an embodiment of the receiver member of the embodiment illustrated in FIGS. 1A through 2B.

FIG. 3C is a sectional view of the receiver member illustrated in FIG. 3A, taken along the lines 3a-3a in FIG. 3A, and viewed in the direction of the arrows.

FIGS. 4A and 4B are front and side views of an embodiment of the bone anchor of the embodiment illustrated in FIGS. 1A through 2B.

FIGS. 4C and 4D are front and side sectional views of the bone anchor illustrated in FIGS. 4A and 4B, taken along the lines 4b-4b of FIG. 4B and viewed in the direction of the arrows.

FIGS. 4E and 4F are enlarged front and side views of the head of the bone anchor illustrated in FIGS. 4A through 4D.

FIGS. 5A and 5B are top and side views of the retaining member illustrated in FIGS. 1 and 2.

FIGS. 5C and 5D are front and side views of an external sleeve for the retaining member illustrated in FIGS. 1 and 2.

FIGS. 6A and 6B are front and side sectional views of the assembled embodiment illustrated in FIGS. 1A through 2B.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein, being contemplated as would normally occur to one skilled in the art to which the invention relates.

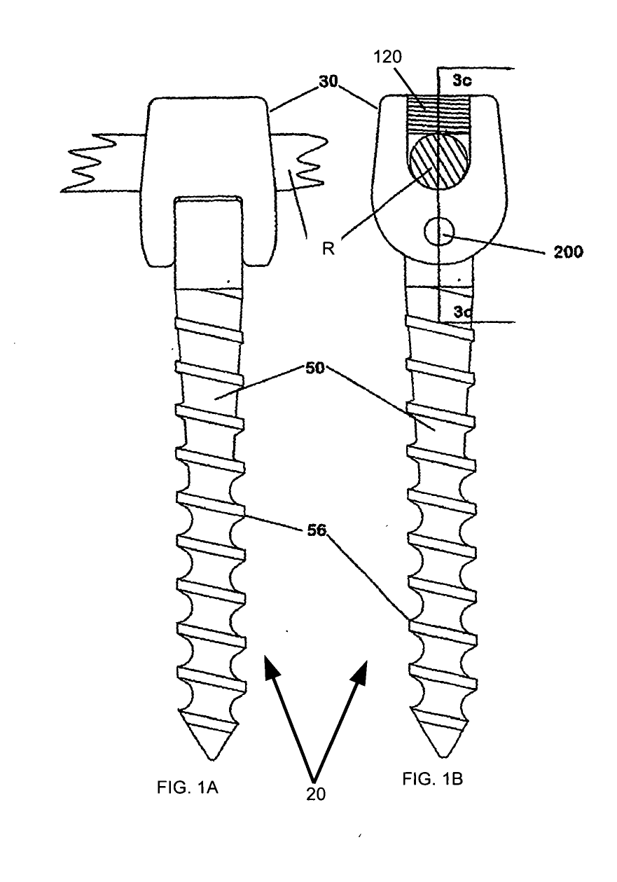

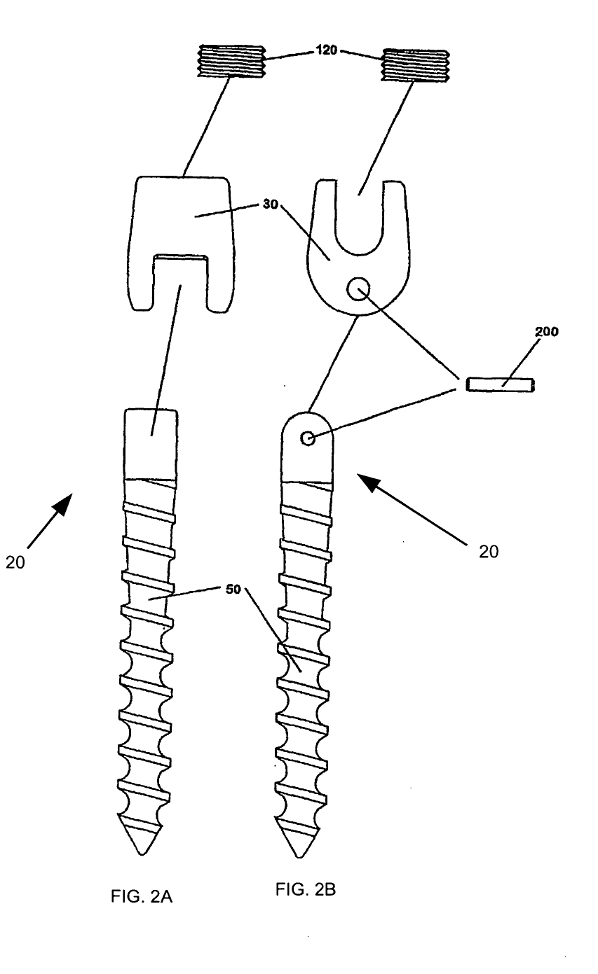

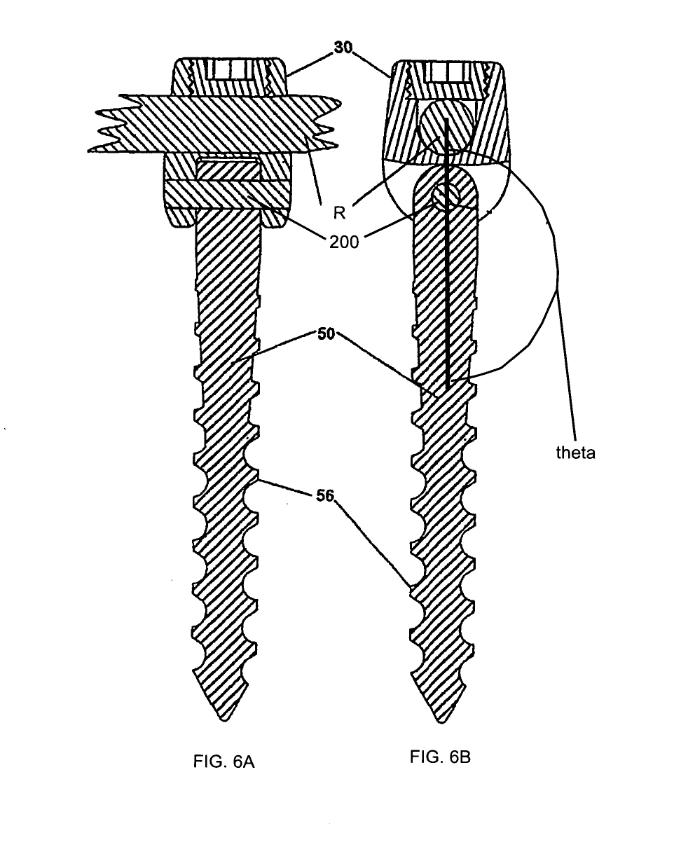

Referring generally to FIGS. 1A, 1B, 2A, 2B, 6A, and 6B, there is shown one embodiment of a single-axial/hybrid jointed bone anchor assembly 20 of the present invention. In the illustrated embodiment, assembly 20 includes a receiver member 30, and a bone anchor 50. The assembly 20 of the present invention is designed for use with an elongated member R such as a spinal rod, bar or other orthopedic construct, as further described below.

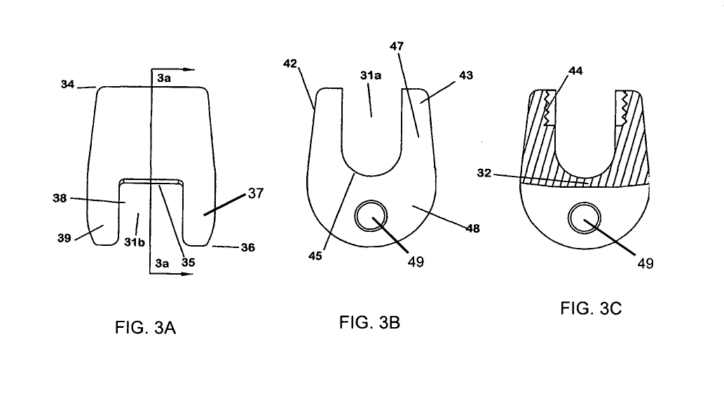

Receiver member 30 (one illustrative embodiment of which is depicted in more detail in FIGS. 3A through 3C) defines an upper opening portion 31a and a lower opening portion 31b, which do not form a single opening in the depicted embodiment. Upper opening portion 31a includes a pair of upright branches 42 and 43 which extend from a top end 34 defining a U-shaped channel 45 therebetween extending from an upper aperture 33 in top end 34 to a floor 32. Internal threads 44 may be formed in the interior surfaces of branches 42 and 43. Internal thread 44 may be a modified acme buttress thread. The top portion 47 of receiver member 30 (which includes branches 42, 43) may be narrower than bottom portion 48 of receiver member 30, thereby reducing the bulk and profile of receiver member 30.

Bottom portion 48 of receiver member 30 includes a lower aperture 35 in bottom end 36 leading to a chamber/void defined by branched chamber walls 37 and 39 to define the bone anchor interface channel 38. As depicted, bone anchor interface channel 38 may be disposed transverse to the U-shaped channel 45, to allow for single axial positioning around an axis parallel to an elongated member placed within U-shaped channel 45. Two connection pin insertion holes 49 may be disposed opposite one another in each of branched chamber walls 37 and 39 to allow for insertion of a connection pin 200 therethrough to span across bone anchor interface channel 38, as discussed in additional detail below.

It will be appreciated that the receiver member 30 may be separated into upper and lower opening portions 31a and 31b at or below the floor 32. Additionally, upper and lower opening portions 31a, 31b can have a variety of configurations, such as each having one or more sections of differing diameter.

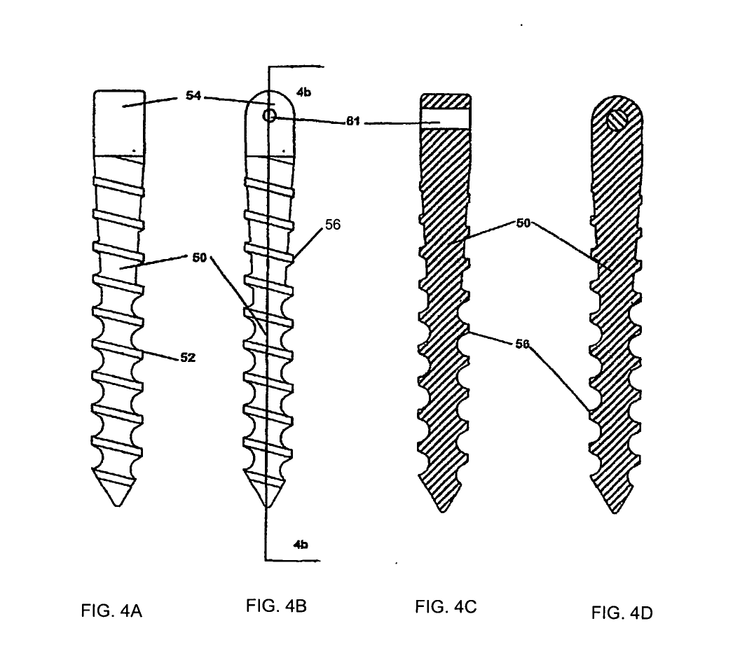

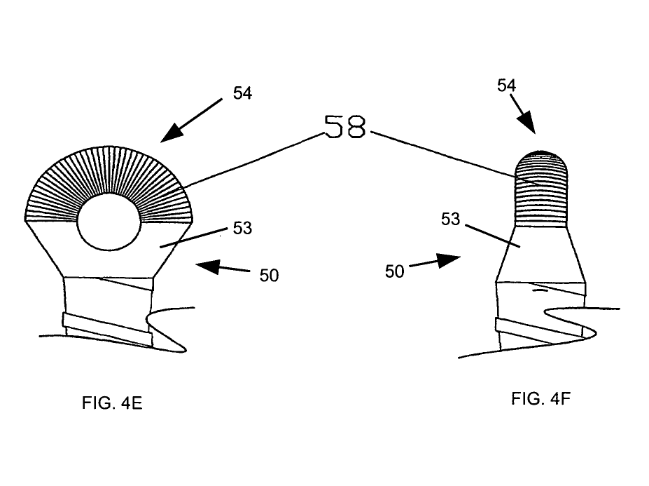

In the depicted embodiments, bone anchor 50 is a bone screw (one illustrative embodiment of which is depicted in more detail in FIGS. 4A through 4D). Bone anchor 50 includes an anchorage portion 52 and a head portion 54. Anchorage portion 52 may be a shaft which includes at least one thread 56, which may be a cancellous self-tapping thread. Other embodiments of bone anchor 50 are contemplated as being within the scope of the present invention. For example, bone anchor 50 could be a bone-engaging hook rather than a screw. In such embodiments, anchorage portion 52 may be configured with a hook rather than a threaded elongated shaft.

Some details of head portion 54 of bone anchor 50 are best depicted in FIGS. 4E and F, which show front and side views of a second illustrative embodiment of a bone anchor head 54. As depicted, head 54 forms part of a cylinder in the illustrated embodiment, though alternative curvate and other configurations may be employed. A narrowing neck 53 may transition from shaft to the head 54. Head 54 includes a through-hole 61, which align with pin insertion holes 49, upon insertion into bone anchor interface channel 38, and through which a pin may be engaged to provide anchorage to the receiver member 30. In one embodiment, the upper portion 58 of head 54 may be polished to a high degree of smoothness to facilitate the movement of the bone anchor 50 throughout the single-axial range.

Head 54 of bone anchor 50 is shaped and sized to fit within the bone anchor interface channel 38 interior void of receiver member 30. Specifically, head 54 has a width that is smaller than the width of lower aperture 35. As more fully described below, bone anchor 50 is inserted into receiver member 30, and retained with pin 200.

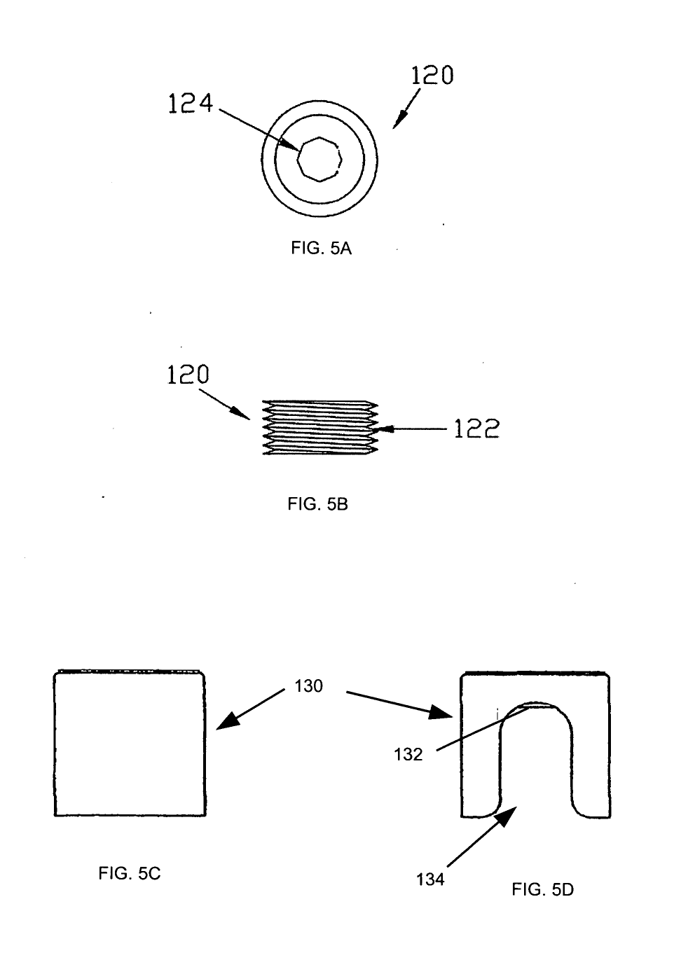

FIGS. 5A and 5B depict one illustrative embodiment of a compression or retaining member 120 in accordance with the principles of the present invention. As depicted, retaining member 120 may be a set screw or threaded plug having external threads 122 and a print 124 for interaction with a tool (not shown) for applying torque. In assembly, retaining member 120 may be threaded into threads 44 of receiver member 30 and down onto an inserted elongated member R. In one alternative embodiment, where receiver member 30 is externally threaded, retaining member 120 could be an internally-threaded nut.

FIGS. 5C and 5D depict one embodiment of an external sleeve 130 which may be used with retaining member 120 in some embodiments of the present invention. Where used, the external sleeve 130 may be placed over the receiver member 30, after an elongated member R, such as a rod, has been inserted with opposite side recesses 134 aligning with the upper channel and the elongated member R passing therethrough. The retaining member 120 may be threadably inserted into the receiver member 30 by rotation of the threads 122 with threads 44 in the branches through a threaded opening or flange 132 disposed in the top surface of the external sleeve 130. A portion of the threaded retainer 120 may reside in the flange 132 after tightening to secure the sleeve 130 to the remainder of the assembly.

Generally referring to FIGS. 1A, 1B, 2A, 2B, 6A and 6B, assembly 20 is assembled as follows: bone anchor 50/pin 200, are inserted into receiver member 30 through bottom end 36, either individually or substantially in one step. Bone anchor 50 remains single-axially moveable with respect to receiving member 30. Head 54 of bone anchor 50 is supported by way of the pinning to the lower portion of the receiver member 30. Assembly 20 may be assembled to this point prior to use in a surgical procedure.

Bone anchor 50 of assembly 20 may be threaded into an appropriately prepared hole in a bone (not shown). It will be understood that in alternative embodiments of the invention, for example where bone anchor 50 is a bone hook, drilling a hole in bone and threading the anchor therein may not be necessary, instead other appropriate attachment protocols may be used. Threaded anchoring portion 52 is inserted into the hole, and an appropriate screwing tool is inserted into the assembly 20, locking the assembly 20 into a single co-axial unit. At this point, the bone anchor 50 is threaded into the bone. When bone anchor 50 has been threaded into the bone to the desired depth, the appropriate screwing tool is removed, and receiver member 30 is positioned so that opening 32 forms a desired angle with bone anchor 50. In the illustrated embodiment, the angle theta between bone anchor 50 and opening 32 can be any value up to about 90 degrees in any direction (up to about 180 degrees total angulation).

As described above, receiver member 30 may be angled as the surgeon desires with respect to bone anchor 50, although because of the design, one each of each configuration must be used on either side of each vertebral body to provide the necessary “locking” of the total construct. An elongated member R such as a spinal rod, connector, or other orthopedic surgical implant may be coupled with assembly 20. Elongated member R is placed in channel 45 of receiver member 30 and a retainer member 120, such as a set screw or threaded plug, is threaded into threads 44 of receiver member 30 and down onto elongated member R. Compression member 120, in one embodiment, is a set screw or plug having external threads 122 and a print 124 for applying torque. In a further embodiment, alternatively, where receiver member 30 is externally threaded, compression member 120 could be an internally-threaded nut. As compression member 120 is tightened, elongated member R is forced downward against floor 32.

The bone anchor 50 is capable of single-axial positioning throughout a 180 degree range of motion. Generally, the disclosed bone anchor apparatus is designed to include two different axial relationship positioning (about 0 degrees and about 90 degrees apart) of the bone anchor member 50 with respect to the receiver member 30, and specifically, this axial relationship to the axial placement of the elongated rod member R. This elongated member R is placed in the channel of the receiver member 30, contacting the floor 32 of the receiving member. A compression retaining member 120 is applied via the upper opening. The compression retaining member 120 presses down on the elongated member R, which presses down on the floor thus locking the elongated member between the retaining member and the receiver member floor. This single elongated member/receiver member, in its self does not provide the necessary axial “locking” required for design parameters; therefore the need for the second configuration of the apparatus. The necessary “locking” parameter is accomplished by alternatively placing one each, of each apparatus configuration on either side of the spine, per each vertebral body to be fused. This unique apparatus placement “locks” the total apparatus construct into one solid unit.

It will be appreciated that where appropriate and desired, the assembly 20 can be assembled during the surgical procedure.

Components of assembly 20 may be constructed of any surgically acceptable material of sufficient strength to be used to retain elongated member R. For example, stainless steel, titanium, and their alloys can be used. It will be appreciated that any sturdy biocompatible material may be used to accomplish the osteosynthesis and other orthopedic surgical goals of the present invention.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. A bone anchor assembly for engagement to an elongated member, comprising:
a receiver member with an upper opening formed as an upper channel between at least two opposite branches and a lower opening comprising a bone anchor interface channel disposed between at least two channel walls having opposite connection pin insertion holes disposed therein;
a bone engaging anchor having a lower portion configured to engage a bone and a head portion having a width smaller than a minimum width of the lower opening of the receiver member allowing the head portion to be movably disposed in the bone anchor interface channel, the head portion further comprising a through hole which aligns with the opposite connection pin holes in the at least two channel walls upon insertion of the head portion into the bone anchor interface channel;
a connection pin configured to press through the head portion of the bone engaging anchor and the opposite connection pin insertion holes of the receiver member to retain the bone engaging anchor in the bone interface channel, such that the bone engaging anchor is capable of single-axial positioning with respect to the receiver member throughout a range of motion of about 180 degrees; and
a compression retaining member for retaining an elongate member within the upper channel of the receiver member.

2. The bone anchor assembly of claim 1, wherein a long axis of the upper channel and a long axis of the bone interface channel of the receiver member are transverse to one another.

3. The bone anchor assembly of claim 1, wherein the upper channel and the bone interface channel are separated from on another by floor of the upper channel.

4. The bone anchor assembly of claim 3, wherein the compression retaining member retains an elongate member within the upper channel by threadably connecting to the receiver member to compress the elongate member against the floor of the upper channel.

5. The bone anchor assembly of claim 4, wherein the compression retaining member is a threaded plug which retains an elongate member within the upper channel by threadably connecting to internal threads disposed on the at least two opposite branches.

6. The bone anchor assembly of claim 1, wherein the head portion of the bone-engaging anchor is at least partially cylindrical.

7. The bone anchor assembly of claim 1, wherein the head portion of the bone-engaging anchor is smooth to facilitate movement within the bone anchor interface channel at the bottom of the receiver member.

8. The bone anchor assembly of claim 7, wherein the bone anchor interface channel at the bottom of the receiver member is at least partially cylindrical to facilitate movement of the head portion of the bone anchor member.

9. The bone anchor assembly of claim 7, wherein the bone anchor interface channel has smooth surfaces to facilitate movement of the head portion of the bone anchor member.

10. The bone anchor assembly of claim 1, further comprising an external sleeve for extending around and over the receiver with a top opening for insertion of the compression retaining member.

11. A spinal fixation assembly comprising:
an elongate member;
a first bone anchor assembly comprising
a first receiver member with an upper opening formed as an upper channel between at least two opposite branches and a lower opening comprising a bone anchor interface channel disposed between at least two channel walls having opposite connection pin insertion holes disposed therein,
a first bone engaging anchor having a lower portion configured to engage a bone and a head portion having a width smaller than a minimum width of the lower opening of the first receiver member allowing the head portion to be movably disposed in the bone anchor interface channel, the head portion further comprising a through hole which aligns with the opposite connection pin holes in the at least two channel walls upon insertion of the head portion into the bone anchor interface channel,
a first connection pin configured to press through the head portion of the first bone anchor and the opposite connection pin insertion holes of the first receiver member to retain the first bone engaging anchor in the bone interface channel, such that the first bone engaging anchor is capable of single-axial positioning with respect to the receiver member throughout a range of motion of about 180 degrees; and
a first compression retaining member for retaining the elongate member within the upper channel of the first receiver member at a first position along the elongate member;
at least a second bone anchor assembly comprising
at least a second receiver member with an upper opening formed as an upper channel between at least two opposite branches and a lower opening comprising a bone anchor interface channel disposed between at least two channel walls having opposite connection pin insertion holes disposed therein,
at least a second bone engaging anchor having a lower portion configured to engage a bone and a head portion having a width smaller than a minimum width of the lower opening of the at least a second receiver member allowing the head portion to be movably disposed in the bone anchor interface channel, the head portion further comprising a through hole which aligns with the opposite connection pin holes in the at least two channel walls upon insertion of the head portion into the bone anchor interface channel,
at least a second connection pin configured to press through the head portion of the first bone anchor and the opposite connection pin insertion holes of the at least a second receiver member to retain the at least a second bone engaging anchor in the bone interface channel, such that the at least a second bone engaging anchor is capable of single-axial positioning with respect to the receiver member throughout a range of motion of about 180 degrees; and
at least a second compression retaining member for retaining the elongate member within the upper channel of the at least a second receiver member at a second position along the elongate member.

12. The spinal fixation assembly of claim 11, wherein the first bone anchor assembly is secured to the elongate member at the first position and the at least a second bone anchor assembly is assembly comprising at least a second receiver member, at least a second bone-engaging anchor is secured to the elongate member at a second point, such that the first bone engaging anchor and the at least a second bone engaging anchor may be secured to either side of a rear surface of a vertebral body.

13. The spinal fixation assembly of claim 11, wherein a long axis of the upper channel and a long axis of the bone interface channel of the first receiver member are transverse to one another.

14. The spinal fixation assembly of claim 11, wherein the upper channel and the bone interface channel of the first receiver member are separated from one another by a floor of the upper channel.

15. The spinal fixation assembly of claim 14, wherein the first compression retaining member is a threaded plug which retains the elongate member within the upper channel of the first receiver member by threadably connecting to internal threads disposed on the at least two opposite branches of the first receiver member to compress the elongate member against the floor of the upper channel.

16. The spinal fixation assembly of claim 11, wherein the head portion of the first bone-engaging anchor is at least partially cylindrical.

17. The spinal fixation assembly of claim 11, wherein the head portion of the first bone-engaging anchor is smooth to facilitate movement within the bone anchor interface channel at the bottom of the first receiver member.

18. The spinal fixation assembly of claim 17, wherein the bone anchor interface channel at the bottom of the receiver member is at least partially cylindrical and has smooth surfaces to facilitate movement of the head portion of the bone anchor member.

19. The spinal fixation assembly of claim 11, further comprising an external sleeve for extending around and over the first receiver with a top opening for insertion of the first compression retaining member.
